(19) United States
(12) Patent Application Publication
(10) Pub. No.: US 2005/0089842 A1
(43) Pub. Date: Apr. 28, 2005

(54) Method for photo-immobilizing and/or recovering a biomaterial

(75) Inventors: Yuichiro Nakaoki, Kisarazu-shi (JP); Osamu Watanabe, Nagoya-shi (JP); Taiji Ikawa, Aichi-ken (JP); Fumihiko Hoshino, Bisai-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AISIN SEIKI KABUSHIKI KAISHA, Kariya-shi (JP)

(21) Appl. No.: 10/941,997

(22) Filed: Sep. 16, 2004

(30) Foreign Application Priority Data
Sep. 17, 2003 (JP) 2003-324099

(51) Int. Cl. C12Q 1/70; C12Q 1/68; G01N 33/554; G01N 33/569; C12M 1/34; G03C 5/04

(52) U.S. Cl. 435/5; 435/6; 435/7.32; 435/287.2; 430/494

(57) ABSTRACT

The present invention provides an advantageous method for immobilizing, analyzing and recovering a biomaterial, and a surface plasmon resonance sensor utilizing the advantage of the method. The method comprises processes of placing a biomaterial on a surface of a carrier having a predetermined photo-immobilizing material, immobilizing the biomaterial by photo-irradiation, and carrying out detection, utilization, analysis or formation of a complex, followed by isolating the biomaterial by subsequent photo-irradiation and by applying external mechanical force to recover the biomaterial or the complex with maintaining the activity or the function thereof. The surface plasmon resonance sensor comprises a photo-immobilizing carrier, a photo-irradiation system, a surface plasmon measurement system and a means for applying moderate external mechanical force.

### CROSS REFERENCE TO RELATED APPLICATIONS

This application is based on and claims priority under 35 U.S.C. § 119 to Japanese Patent Application 2003-324099, filed on Sep. 17, 2003, the entire content of which is incorporated herein by reference.

### BACKGROUND OF THE INVENTION

1. Field of the Invention

The present invention relates to a method for photo-immobilizing and/or recovering a biomaterial and a surface plasmon resonance sensor, and more specifically, to a method for photo-immobilizing and/or recovering a biomaterial by immobilizing a variety of extremely small biomaterials on a surface of a carrier using an optical means, and also detecting, utilizing or analyzing the immobilized biomaterial or the complex thereof depending on the purpose, followed by isolating the immobilized biomaterial or the complex thereof to recover it from the carrier surface using mild manipulation without damaging the immobilized biomaterial or the complex thereof, and a surface plasmon resonance sensor implementing the above method which can recover a sample while maintaining the activity or the function thereof.

2. Description of the Related Art

In recent years, nanotechnology for analyzing or constituting extremely small subjects, has been drawing attentions in the technical fields such as in the field of materials science and mechanics. Particularly, in the field of biotechnology, results from numerous researches applying nanotechnology to molecular biology have often been reported.

For example, with the advancement in molecular biology, researches for analyzing polynucleotides such as genes, various polypeptides expressed by genes (an enzyme, an antigen-antibody, a cell membrane receptor protein, etc.), saccharide chains, etc. have been drawing a considerable amount of attention. Accordingly, in vitro analysis using organelles, cells, microorganisms, etc. is very important in molecular biology.

Further, one of the central issues in the medical field of the post-genome era is gene diagnosis or DNA diagnosis. Specifically, by analysis of restriction fragment length polymorphism (RFLP) or a DNA fragment comprising microsatellite parts, or by analysis of single nucleotide polymorphisms (SNPs), so-called genome-based drug discovery can be carried out, or by identifying genetic information of individuals, tailor-made therapies and provision of expert witness in legal medicine can be performed.

In view of the above, it is desirable to have a technology which makes it possible to selectively immobilize various micro biomaterials such as the above-mentioned polynucleotides, polypeptides, organelles and cells in their active or viable states, to conduct utilization, analysis or the like of the biomaterials in various forms such as in a complex and further preferably to recover the biomaterials or a complex thereof while maintaining their active or viable states.

As an important sensor that is used for such purpose, there is a surface plasmon resonance sensor based on surface plasmon resonance (SPR). The SPR method utilizes SPR phenomenon wherein a light beam incident on a thin metallic film having a thickness of, for example, 100 nm or less under a total reflection condition is converted to a surface wave in the thin metal film by resonance at a particular angle of incidence. The angle that generates SPR varies sensitively depending on the change of refractive index around the metal, and the intensity of the reflected light is reduced because the energy from the incident light is used to excite SPR. Accordingly, if a functional protein, etc. immobilized on a carrier surface is specifically bound to a subject to be analyzed, refractive index change occurs, which can be detected sensitively.

[Patent Document 1] JP-A-2003-116515

In Patent Document 1, the section [0005] of the specification discloses, as a description of a prior art, that a prism constituting a surface plasmon resonance sensor is modified by an antibody, and when an antigen is bound by the antibody, after its detection, the bio-molecule can be dissolved out by flowing a buffer solution with varied pH or salt concentration.

Accordingly, this description of the prior art discloses a technique in which a bio-molecule as an antigen is immobilized by a carrier in the form of an antigen-antibody complex, and then the bio-molecule is recovered by dissociating the antigen-antibody complex.

The invention of Patent Document 1 discloses a technique in which the fine particles serving as a probe is immobilized on a substrate, a bio-molecule to be analyzed is adsorbed on the fine particles, interaction of bio-molecules is detected, and then the bio-molecule is recovered inclusive of the fine particles. A technique of irradiating a laser pulse is also disclosed as a technique for recovering the fine particles.

Accordingly, these disclosures can be understood to disclose a technique with which a bio-molecule is immobilized on the fine particles on a substrate and after the detection of the interaction of bio-molecules the bio-molecule is recovered inclusive of the fine particles, and a recovering means thereof.

### SUMMARY OF THE INVENTION

Namely, Patent Document 1 discloses a technique for immobilizing and recovering protein or DNA of a bio-molecule by utilizing an immune reaction in the technique described as a prior art in Patent Document 1, or by utilizing, e.g., DNA hybridization as described in the invention of Patent Document 1. However, since this immobilizing means is forming of a complex (an antigen-antibody complex or a DNA hybrid chain) and one of the bio-molecules which constitute the complex should be immobilized in advance, the prior arts have the disadvantages stated below in recovering the immobilized bio-molecule or a complex thereof.

Specifically, firstly, since one of the bio-molecules that constitute a complex (an antigen or a DNA probe) cannot be isolated or recovered from the carrier (the prism or the fine particles), a considerable loss in cost may be generated if such a bio-molecule is valuable or expensive. Secondly, for a bio-molecule which is obtained by isolation from the carrier, especially for an antibody, in order to recover by maintaining activity of the antibody, the means or conditions for recovering it are required to be investigated separately, thus entails complicate procedures. Thirdly, the recovery of a bio-molecule (an antigen or a complementary chain DNA) requires dissociation of the bio-molecules which constitute a complex (an antigen-antibody complex, or a hybrid chain), thus making it impossible to recover the complex itself for analysis, etc.

Therefore, an object of the present invention is to provide a method for immobilizing a variety of extremely small biomaterials on a surface of a carrier using an optical means, and also for recovering the immobilized biomaterials by simple and mild recovering means without damaging the biomaterials, and further a method for recovering a complex having the immobilized biomaterials in a similar manner. Another object of the present invention is to provide a surface plasmon resonance sensor implementing the same method which can recover a sample with maintaining the activity or the function thereof.

Specifically, the present invention provides a method for photo-immobilizing and/or recovering a biomaterial, wherein the method comprises (a) a process of immobilizing the biomaterial by placing the biomaterial on a surface of a carrier having a photo-immobilizing material which is plasticized by photo-irradiation and have immobilization ability by photo-modification at least in a surface layer part, and (b) a process of recovering the immobilized biomaterial by isolating the immobilized biomaterial from the carrier surface by photo-irradiation and by applying external mechanical force.

The present invention further provides a surface plasmon resonance sensor comprising (a) a carrier whose surface layer part is provided with a layer of a photo-immobilizing material plasticized by photo-irradiation and has immobilization ability by photo-modification for a micro-object placed on the surface, and with a thin layer of a metal film placed below the layer of the photo-immobilizing material, (b) a photo-irradiation system having the constitution for photo-irradiating on the surface of the carrier, (c) a surface plasmon measurement system for detecting a refractive index change of the surface layer part of the carrier by a certain reaction of biomaterials on the surface of the carrier, and (d) a means for applying external mechanical force on the surface of the carrier.

(First Aspect of the Present Invention)

The first aspect of the present invention to achieve the above object is to provide a method for photo-immobilizing and/or recovering a biomaterial, which comprises a process of immobilizing the biomaterial on the carrier surface by photo-irradiation, preceded by placing the biomaterial of (B) as described below on a surface of a carrier having the photo-immobilizing material of (A) as described below at least in a surface layer part, and a process of recovering the immobilized biomaterial by isolating the immobilized biomaterial from the carrier surface by photo-irradiation and by applying external mechanical force:
(A) photo-immobilizing material: a material which is plasticized by photo-irradiation and has immobilization ability by photo-modification, for a micro-object which is placed on the surface; and
(B) biomaterial: polypeptide, saccharide chain, polynucleotide, organelle, cell, bacteria, virus or a combination thereof.

In the first aspect, “photo-modification” includes modification by binding of a biomaterial with the carrier surface by a motion on the molecular level, etc. in addition to modification in a usual and macroscopic sense. Such photo-modification may be observed clearly by an optical microscope, an electron microscope, or the like in some instances, but it may not be observed clearly by the conventional observation means depending on the amount and form of the modification in other instances.

(Second Aspect of the Present Invention)

The second aspect of the present invention to achieve the above object is to provide a surface plasmon resonance sensor comprising (a) to (d) as below:
(a) a carrier whose surface layer part is provided with a layer of a photo-immobilizing material which is plasticized by photo-irradiation and has immobilization ability by photo-modification for a micro-object placed on the surface, and with a thin layer of a metal film placed below the layer of the photo-immobilizing material,
(b) a photo-irradiation system having the constitution for photo-irradiating on the surface of the carrier,
(c) a surface plasmon measurement system for detecting a refractive index change of the surface layer part of the carrier by a certain reaction of biomaterials on the surface of the carrier, and
(d) a means for applying external mechanical force on the surface of the carrier.

### BRIEF DESCRIPTION OF THE DRAWINGS

FIG. 1 briefly shows a biomaterial recovery instrument related to Examples.

FIG. 2 briefly shows an SPR measurement-recovery complex instrument related to Examples.

### DESCRIPTION OF THE PREFERRED EMBODIMENTS

The present inventors have studied a means to achieve the above-mentioned objects, and thus have found that if a biomaterial is placed on the surface of “a photo-immobilizing material which is plasticized by photo-irradiation and has immobilization ability by photo-modification, for a micro-object which is placed on the surface” and if photo-irradiation is carried out, the biomaterial is strongly immobilized on the surface of the photo-immobilizing material. Further, they have also found that if photo-irradiation is further carried out on the immobilized biomaterial again or repeatedly, the immobilization force is weakened to some extent at each photo-irradiation, and the biomaterial can be isolated from the surface of the material by applying external mechanical force such as by flowing a liquid which contacts with the surface of the material at some flow rates.

In these phenomena, it is understood that the photo-immobilizing material is plasticized by photo-irradiation, and modified depending on the shape of the biomaterial (for example, modification into the concavo-convex shape corresponding to the shape of the biomaterial) to obtain semi-immobilized state in which the biomaterial is immobilized at some strength, and then to obtain complete immobilized state in which the biomaterial is strongly immobilized by returning from plasticized state to solidified state after completing the photo-irradiation. The strong immobilization force in the complete immobilized state is considered to be due to the effects of supporting the biomaterials by the photo-modified surface of the material, the enhanced adhesion force such as the van der Waals interaction by increase in the contact area between the surface of the material and the biomaterial, etc.

Then, if photo-irradiation is further carried out on the immobilized biomaterial again or repeatedly, the photo-immobilized material is plasticized at each photo-irradiation and the immobilization state for the biomaterial returns from the complete immobilized state to the semi-immobilized state. In addition to this, if external mechanical force (external mechanical force which does not damage the active or viable state of the biomaterial) is applied, the biomaterial can be isolated.

In the method for photo-immobilizing and/or recovering a biomaterial, extremely small biomaterials such as polypeptide, saccharide chain, polynucleotide, organelle, cell, bacteria, virus or a combination thereof can be easily photo-immobilized and recovered.

In addition, since a biomaterial can be photo-immobilized and recovered by an easy and mild means, such as by photo-irradiation and by applying moderate external mechanical force, a means for carrying out the invention and a method for carrying out the invention are unusually simple and require low cost while they enable the biomaterial to be photo-immobilized and/or recovered in their active or viable state. Activity loss or change of the steric shape of biomaterials such as enzyme can be prevented, destruction of DNA having a weak chemical structure can be prevented, and the viable state of cells or bacteria can be well maintained. Accordingly, a biomaterial which is first immobilized to a carrier, can be recovered in the free state for any purpose, and further the function, activity, etc. thereof can be re-evaluated.

It is preferred that an intermediate process of detecting, utilizing or analyzing the immobilized biomaterial is carried out between the immobilizing process and the recovering process. Accordingly, various biomaterials can be provided for desired manipulation in the immobilized state which is suitable for detection, utilization or analysis, and after that, easily recovered in their active or viable states.

The intermediate process may comprise the formation of a complex based on specific adsorption or reaction between the biomaterials. The complex comprises a complex of one kind of a second biomaterial and an immobilized biomaterial, or a complex formed by specifically binding plural kinds of a second biomaterial sequentially to an immobilized biomaterial. The complex as a whole is recovered by isolation from the carrier surface.

In other words, conventionally, a complex formed by an immune reaction such as an antigen-antibody reaction was not easily isolated and recovered with high purity and efficiency from antigens or antibodies which have not formed a complex yet. However, the complex can be easily isolated and recovered with high purity in the recovering process. The function and the activity of the thus-isolated and recovered complex are not inhibited (for example, the complex maintains the original steric conformation) and therefore the complex can be analyzed using a means such as a mass spectrometric analysis, NMR and XRD.

In addition, in the intermediate process, it is also possible that the formed complex is once dissociated, and the second biomaterial is first recovered, and then the biomaterial immobilized on the carrier is recovered. Therefore, the present invention has no disadvantage in that all of the biomaterials which constitute the complex can be recovered, and it does not generate a considerable loss in cost, in the case wherein the bio-material is valuable or expensive.

By carrying out formation of a complex and detection for the complex in the intermediate process, the method for photo-immobilizing and/or recovering a biomaterial can be applied to various uses such as a sample for a bio-reactor, a bio-sensor and a bio-assay, and a protein chip for a proteome analysis.

Detection of the complex is carried out, for example, by a marker provided in a second biomaterial which constitutes the complex.

Detection of the complex is also carried out, for example, by the principle making use of SPR in which a carrier is provided with a thin metal film layer in a predetermined constitution.

In other words, this is a surface plasmon resonance sensor. The surface plasmon resonance sensor has a great merit in that a second biomaterial for detection or a complex itself can be recovered in its active or viable state.

The kind of photo-immobilizing material is not limited if it corresponds to the definition, but it is particularly preferable that it is a material possessing a chemical structure capable of causing cis-trans optical isomerization by photo-irradiation. In such material, the motion on the molecular level by cis-trans optical isomerization plasticizes the photo-immobilizing material, thus allowing easy photo-modification.

The “chemical structure” is particularly preferably a pigment structure having an azo group. A pigment structure having an azo group (especially, an azobenzene structure) has a particularly prominent effect of plasticizing the photo-immobilizing material based on the motion on the molecular level by cis-trans optical isomerization, thus allowing easy photo-modification.

The photo-irradiation in the immobilizing process and the recovering process is preferably carried out using light of a low photon energy (for example, visible light). If light of a high photon energy (for example, ultraviolet light) is used, it may cause activity loss or damage of a biomaterial to be immobilized. A specific standard of the preferred photon energy varies depending on the kind of biomaterial to be immobilized, the environment of photo-irradiation, etc. and therefore the standard cannot be prescribed uniformly.

Further, the photo-irradiation in the immobilizing process and the recovering process is preferably carried out using light of such a power that elevation of temperature around the biomaterial is maintained within a temperature range which does not damage the function of the biomaterial. Since a specific standard of the preferred power of light varies depending on the kind of biomaterial to be immobilized, the environment of photo-irradiation or the like, the standard cannot be prescribed uniformly.

Since the surface plasmon resonance sensor comprises each element of a carrier of (a), a photo-irradiation system of (b), a surface plasmon measurement system of (c), and a means for applying a load of (d), the second biomaterial for detection or a complex itself can be recovered in its active or viable state. In addition, the carrier relating to immobilization of a biomaterial, and formation and recovery of a complex, the photo-irradiation system and the means for applying the load can be constituted inexpensively and easily.

Embodiments for carrying out the inventions according to these aspects will be explained hereinbelow including preferred embodiments. “The present invention” simply mentioned in the following indicates each of the aspects of the present invention collectively.

Method for Photo-Immobilizing and/or Recovering a Biomaterial

A method for photo-immobilizing and/or recovering a biomaterial according to the present invention comprises a process of immobilizing the biomaterial on the carrier surface by photo-irradiation, preceded by placing the biomaterial on a surface of a carrier having a photo-immobilizing material plasticized by photo-irradiation at least in a surface layer part, and a process of recovering the immobilized biomaterial by isolating the immobilized biomaterial from the carrier surface by photo-irradiation and by applying external mechanical force (usually, moderate external mechanical force) to recover it.

The above-mentioned carrier is not limited in the form, quality, use, etc. in so far as the carrier is made of a photo-immobilizing material, or the carrier is one having the photo-immobilizing material at least in a surface layer part of a substrate with an inorganic or organic material. Forms of the carrier include, for example, a form of relatively small chips such as a DNA chip, a form of particles for filling in column, a form of relatively big immobilization reaction beds, a form such as a test paper used in a simple immunoassay, a form such as a slide glass used in microscope observation, etc. In addition, any constituting element can be added depending on the purpose of the carrier. For example, a thin layer of a metal film is provided under the layer of the photo-immobilizing material in a surface layer part of the carrier as described below.

“Placing” of the biomaterial means placing the biomaterial as contacted or contactable with the carrier surface. For this purpose, for example, the biomaterial is stably contacted with the carrier surface using the affinity between the photo-immobilizing material and the biomaterial, or the photo-irradiation can be carried out with the carrier soaked in water or a buffer solution containing the biomaterial, or the photo-irradiation can be carried out with dropping a small amount of water or a buffer solution containing the biomaterial onto the carrier surface. If the photo-immobilizing material is a polymer material which originally has low hydrophilic property, a hydrophilic group may be introduced to the polymer to increase the affinity with the biomaterial, and therefore the biomaterial can be easily placed on the carrier surface.

“Applying moderate external mechanical force” preferably includes flowing, shaking, or stirring a liquid which contacts with the carrier surface at some flow rates. It also includes adding vibration (for example, applying mechanical vibration or ultrasonic vibration) to the liquid which contacts with the carrier and/or the surface thereof.

In addition, in the method for photo-immobilizing and/or recovering a biomaterial according to the present invention, an intermediate process of detecting, utilizing or analyzing the immobilized biomaterial on the carrier surface can be carried out between the immobilizing process and the recovering process. Further, the intermediate process comprises specifically adsorbing or reacting one kind of a second biomaterial on or with the above-mentioned biomaterial, or specifically adsorbing or reacting plural kinds of a second biomaterial sequentially on or with the above-mentioned biomaterial to form a complex. In the above-mentioned recovering process, the complex can be recovered by isolation from the carrier surface. Alternatively, the complex, once formed and provided for detection, utilization or analysis, is dissociated into units of the biomaterials which are the constituting elements, the dissociated second biomaterial is first recovered, and then the biomaterial immobilized on the carrier is recovered. Further, only the biomaterial immobilized on the carrier can also be recovered.

In the intermediate process, the above-mentioned formation of a complex and detection thereof can also be carried out. In other words, formation of a complex can be carried out for the purpose of detection if a biomaterial which is specifically adsorbed on or reacted with the immobilized biomaterial is contained in the group of the second biomaterials. In this case, the group of the second biomaterial can be provided with a detection marker. The detection marker is preferably a so-called fluorescence label for a biomaterial, though other detection markers such as a label with a radioactive element can also be used. For high sensitivity of detection, an enzyme-linked immunosorbent assay can be used. In addition to fluorescence and RI, chemiluminescence, electrochemiluminescence, absorption spectrum, etc. can also be used as a detection method. In this case, a thin layer of a metal film is provided under the layer of the photo-immobilizing material in a surface layer part of the carrier, and detection of the above-mentioned complex is carried out by detecting a change in the resonance angle of the surface plasmon resonance caused by a refractive index change in the surface layer part of the carrier due to the formation of the complex.

The above-mentioned complex means a complex formed by binding a photo-immobilized biomaterial on the carrier to a second biomaterial that is specifically reacted with or adsorbed on the immobilized biomaterial, or a complex formed by specifically binding one or more kind of second biomaterials sequentially to the second biomaterial. A biomaterial that constitutes the complex may be provided with a detection marker.

The embodiments of the complex are not specifically limited, but include, for example, a hybrid chain in the case where the photo-immobilized biomaterials on the carrier are polynucleotides, an antigen-antibody complex in the case where the photo-immobilized biomaterials on the carrier are antigens (or antibodies), a receptor-ligand complex in the case where the photo-immobilized biomaterials on the carrier are receptor-proteins, a bonding of a signal transduction material with a membrane protein of cells or bacteria in the case where the photo-immobilized biomaterials on the carrier are cells or bacteria, etc. The complex formed by binding plural kinds of a second biomaterial to the photo-immobilized biomaterials on the carrier, is, for example, a complex formed by specifically binding a substrate-decomposing enzyme which further serves as a detection marker, to one of the biomaterials in the antigen-antibody complex formed as described above.

Photo-Immobilizing Material

A photo-immobilizing material in the present invention means a material which is plasticized by photo-irradiation and has immobilization ability by photo-modification for a micro-object (biomaterial) which is placed on the surface. The photo-modification is as defined above.

The photo-immobilizing material is particularly preferably a material possessing a chemical structure capable of causing cis-trans optical isomerization by photo-irradiation. Such chemical structure is particularly preferably a pigment structure having an azo group. The pigment structure having an azo group is preferably a chemical structure of azobenzene or a derivative thereof.

One of preferred examples of the photo-immobilizing material is a photo-immobilizing material containing a component possessing a chemical structure capable of causing cis-trans optical isomerization by photo-irradiation (hereinafter, such component may be also referred to as a photo-reactive component) in a material which is a matrix. The photo-reactive component may be simply dispersed in the matrix material, or chemically bonded or hydrogen-bonded to the matrix material, but the latter is particularly preferred. The matrix material may be an organic material such as conventional polymer materials, or an inorganic material such as glass. The polymer material is particularly preferred.

The kind of polymer material is not limited, but a polymer comprising a urethane group, a urea group or an amide group in the repeating units is preferred in view of heat-resistance. This is based on the following reasoning: The photo-immobilizing material of the present invention is preferably plasticized by intentional photo-irradiation, but not by practically inevitable photo-irradiation of light having a low energy and a low power such as natural light or general indoor illumination, and also temporal stability of such property is required. In view of these points, a glass transition temperature is preferably 100° C. or higher. Needless to say, the photo-immobilizing material having a glass transition temperature of less than 100° C. can also be used.

Preferred examples of a polymer material comprising a photo-reactive component are shown in Formula I to Formula IV as follows.

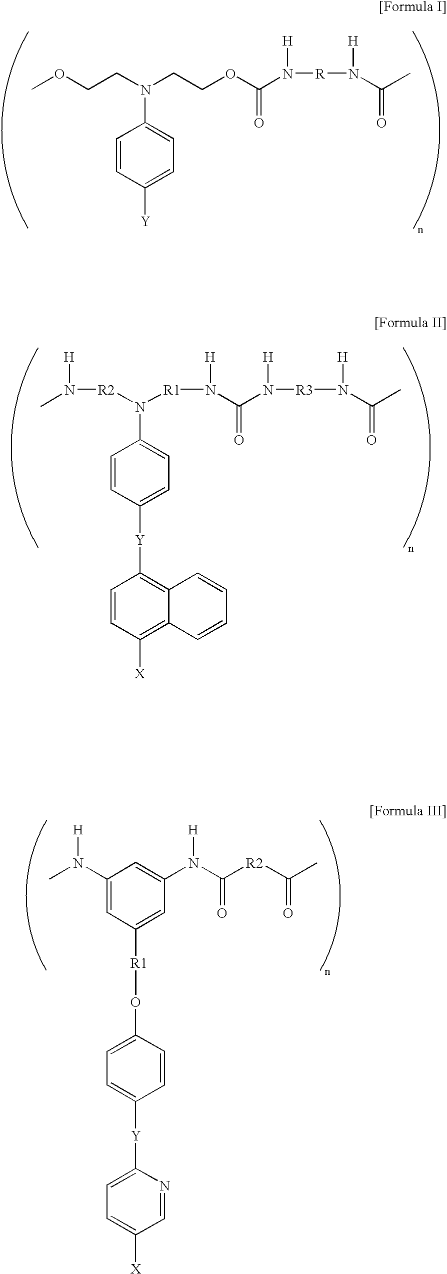

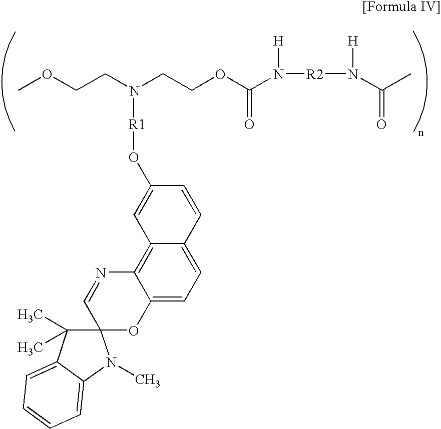

In Formula I through Formula IV, —X represents a nitro group, a cyano group, a trifluoromethyl group, an aldehyde group or a carboxyl group, —Y— represents —N═N—, —CH═N— or —CH—CH—, and —R— represents a phenylene group, an oligomethylene group, a polyethylene group or a cyclohexane group.

Biomaterial

A biomaterial in the present invention means polypeptide, saccharide chain, polynucleotide, organelle, cell, bacteria, virus or a combination thereof.

The polypeptide includes an enzyme, an antigen and an antibody, a cell membrane receptor protein and various other proteins expressed by living cells. The saccharide chain includes N-glucoside-type saccharide chain and O-glucoside-type saccharide chain. The polynucleotide can be exemplified by DNA and/or RNA of a single chain, or double or more chains. More specifically, the polynucleotide includes a DNA fragment comprising single-nucleotide polymorphisms, a DNA fragment comprising a microsatellite part or a restriction fragment, or m-RNA or a fragment thereof, c-DNA or a fragment thereof, a genome DNA fragment, or the like.

The biomaterial may be photo-immobilized after it is placed at a certain site of the carrier surface using laser trapping, etc., or many biomaterials may be photo-immobilized on the carrier surface according to certain distribution patterns by providing a certain distribution to the irradiation region or the irradiation intensity of irradiation light. Only one kind of a biomaterial may be photo-immobilized according to random or certain distribution patterns, or plural kinds of biomaterials may be photo-immobilized according to random or certain distribution patterns, on the surface of a carrier.

Photo-Irradiation

The irradiation light includes any light such as transmission light and near field light or Evanescent light. Light source is not particularly limited, but it is selected from a xenon lamp, LED, a laser and the like. When a laser is used, polarization property thereof can be used. The photo-irradiation is preferably carried out using the light of a low photon energy such as visible light, or the light of such a power that elevation of temperature around the biomaterial is maintained within a temperature range which does not damage the function of the biomaterial.

The method for photo-irradiation is preferably providing certain distributions to the irradiation region or the irradiation intensity of irradiation light as described above. As a means to achieve this, for example, a photo mask can be used, or a finely collimated focused light beam can be used for photo-irradiation according to a certain pattern.

Surface Plasmon Resonance Sensor

A surface plasmon resonance sensor related to the present invention comprises at least the above-mentioned carrier provided with a layer of a photo-immobilizing material and a thin layer of a metal film under the layer of the photo-immobilizing material, a photo-irradiation system with which photo-irradiation can be carried out for the carrier surface as described above, a surface plasmon measurement system which can detect a refractive index change of the surface layer part of the carrier due to the formation of a complex of the biomaterial immobilized on the carrier surface, and the above-mentioned means for applying a moderate external mechanical force on the carrier surface.

In the surface plasmon resonance sensor related to the present invention, the above-mentioned elements may be constituted as an integral complex instrument, or may be constituted independently per element or element group in a mechanical sense while they form a surface plasmon resonance sensor system as a whole.

### EXAMPLES

### Examples 1 to 4

### Photo-Immobilization of a Biomaterial, Formation of a Complex and Recovery of the Complex

### Example 1

### Synthesis of a Photo-Immobilizing Material

Synthesis of a polymeric photo-immobilizing material was carried out according to a conventional method. First, the compound represented by Formula V as shown below was synthesized using a known diazo-coupling method. Then, the compound represented by Formula VI as shown below was synthesized by a known acid chloride reaction. In these compounds, the part of an azobenzene structure constitutes a photo-reactive component.

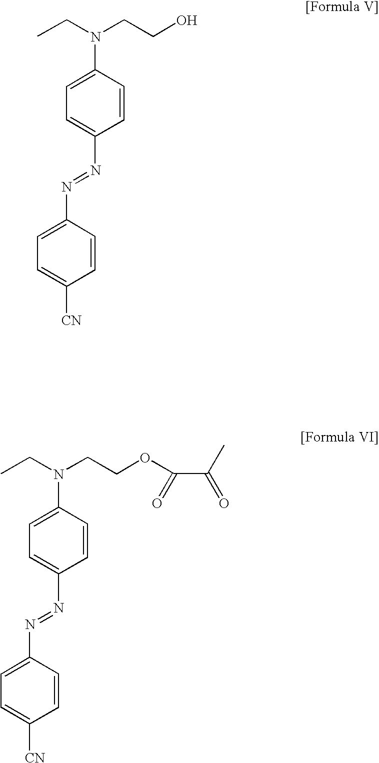

Commercially available methyl methacrylic acid (MMA: Wako Pure Chemical Industries, Co., Ltd.) was prepared, and polymerization inhibitor was removed by distillation under reduced pressure. Then, 0.362 g (0.001 mole) of the compound represented by Formula VI and 0.600 g (0.006 mole) of MMA were copolymerized to synthesize a polymeric photo-immobilizing material.

To a 100 ml egg-plant type flask were added and mixed the above-mentioned monomers, and then added 50 ml of dimethylformamide and 82 mg of 2,2-azoisobutyronitrile. After sealing the flask with a rubber stopper, nitrogen-bubbling was conducted for 1 hour to remove oxygen in the system. Then, the flask was heated for 2 hours at 60° C. with nitrogen-bubbling. Then, the reaction solution was taken out from the flask and was recrystallized with methanol. Such recrystallization was repeated three times, followed by distillation under reduced pressure to give a polymeric photo-immobilizing material of a bi-copolymer related to Example 1.

### Example 2

### Preparation of a Polymeric Photo-Immobilizing Material Film

A polymeric photo-immobilizing material of 12.5 mg of the bi-copolymer related to Example 1 was dissolved in 2 ml of pyridine, and filtered with a filter with a pore diameter of 0.2 μm. The filtrate was dropped on the surface of MAS-coated slide glass (Matsunami Glass Co., Ltd.), and the slide glass was spin-cast at 4000 rpm, to prepare a polymeric photo-immobilizing material film on the slide glass. The thickness of the polymeric photo-immobilizing material film was confirmed to be uniform and about 20 nm by absorption spectrophotometry.

### Example 3

### Antigen Immobilization onto a Photo-Immobilizing Carrier and Antigen-Antibody Reaction

The slide glass on which a polymeric photo-immobilizing material film related to Example 2 was formed, was cut out into a size of 1 cm², to prepare a photo-immobilizing carrier. Onto the surface of the polymeric photo-immobilizing material film in this photo-immobilizing carrier was dropped 1 μl of a phosphate buffer solution containing 0.01 mg/ml of rabbit IgG labeled with Cy3, an indodicarbocyanin-based fluorescence substance as a first biomaterial. After drying off water naturally, light of 10 mW/cm² at a wavelength of 470 nm was irradiated for 30 minutes on the side of the film surface. Immediately after the irradiation, the photo-immobilizing carrier was washed with a phosphate buffer solution, to wash out the IgG which was not immobilized to the polymeric photo-immobilizing material film. The photo-immobilized area of IgG was observed using a fluorescence microscope. As a result, a circular spot with a diameter of about 2 mm derived from Cy3 was found.

Then, onto the film surface of the photo-immobilizing material in the photo-immobilizing carrier was dropped 2 μl of a phosphate buffer solution containing 0.01 mg/ml of a goat-derived anti-rabbit IgG antibody labeled with Cy5, an indodicarbocyanin-based fluorescence substance as a second biomaterial. Then, the photo-immobilizing carrier was kept in a thermostated water bath for 30 minutes at a temperature of 37° C. and a humidity of 85% RH. Then, the photo-immobilizing carrier was washed three times with a phosphate buffer solution. The photo-immobilized area of the goat-derived anti-rabbit IgG antibody was observed using a fluorescence microscope. As a result, a circular spot with a diameter of about 2 mm as described above was found, and this was found to emit both of fluorescence derived from Cy3 and fluorescence derived from Cy5. In other words, confirmed was adsorption of the goat-derived anti-rabbit IgG antibody which is a second biomaterial to rabbit IgG which is a first biomaterial and immobilized onto the photo-immobilizing carrier.

### Example 4

### Recovery of a Biomaterial and Confirmation of Formation of a Complex

Two pieces of the photo-immobilizing carrier in which an antigen and an antibody were immobilized as described in Example 3, were separately put into two transparent plastic tubes with an internal diameter of about 1.2 cm, and into each tube was dropped 1 ml of a phosphate buffer solution. On the side of one of these transparent plastic tubes was placed a blue LED, and the photo-immobilizing carrier in the tube was photo-irradiated by the LED, while stirring using a rotator (Example 4). The other transparent plastic tube was stirred with a rotator under a completely shielded condition with an aluminum foil from light (Comparative Example 4).

The stirring was terminated 24 hours after the initiation of the stirring, and the phosphate buffer solution was recovered from each transparent plastic tube, and the fluorescence from both of the phosphate buffer solutions was detected with a fluorescence spectrometer. As a result, fluorescence spectra of Cy3 and Cy5 were detected in the phosphate buffer solution related to Example 4. No fluorescence spectra of Cy3 and Cy5 were detected in the phosphate buffer solution related to Comparative Example 4.

In addition, when the excitation spectrum of Cy5-derived fluorescence in the phosphate buffer solution related to Example 4 was measured, a peak near about 630 nm belonging to an absorption peak of Cy5 and a peak near about 530 nm belonging to an absorption peak of Cy3 were confirmed. These results suggest that transfer of the excitation energy from Cy3 to Cy5 occurred. Based on Forster's equation which represents the mechanism of the excitation energy transfer, it can be shown that the distance between Cy3 and Cy5 in the system is about 100 nm or less, and therefore the results obtained indicate that a rabbit IgG containing Cy3 and a goat-derived anti-rabbit IgG antibody containing Cy5 are bound to form an antigen-antibody complex.

### Examples 5 to 6

### Detection of an SPR Angle Change Using a Photo-Immobilizing Carrier Chip

### Example 5

### Formation of a Polymeric Photo-Immobilizing Material Film onto the Surface of a Gold Film

Gold was vapor-deposited on a surface of a glass substrate of a predetermined size to form a thin film of gold with a thickness of 50 nm. Then, 6 mg of the polymeric photo-immobilizing material obtained in Example 1 was dissolved in 2 ml of pyridine, and filtered with a filter having a pore diameter of 0.2 μm. The filtrate of 50 μl was dropped on the surface of the above-mentioned thin film of gold, and was spin-cast at 4000 rpm, to give an SPR sensor chip formed by forming a film of the polymeric photo-immobilizing material on the surface of a thin film of gold on the glass substrate. The thickness of the film of polymeric photo-immobilizing material in the SPR sensor chip was confirmed to be 5 nm by reflection absorption spectrophotometry.

Onto the surface of the SPR sensor chip was dropped 1 μl of a phosphate buffer solution containing 0.01 mg/ml of a goat-derived anti-rabbit IgG antibody as a first biomaterial. After drying off water naturally, light of 10 mW/cm² at a wavelength of 470 nm was irradiated for 30 minutes on the side of the film surface. Immediately after the irradiation, the SPR sensor chip was washed with a phosphate buffer solution, to wash out the goat-derived anti-rabbit IgG antibody which was not immobilized to the SPR sensor chip.

### Example 6

### Detection of an SPR Angle Change

An SPR sensor chip in which a goat-derived anti-rabbit IgG antibody was immobilized, was attached to an SPR measurement instrument provided with a surface plasmon measurement system which can detect a refractive index change of the surface layer part of the chip by a certain reaction between the biomaterials on the surface of the chip. Then, 100 μl of a PBS solution (phosphate buffered saline) containing 20 μg/ml of rabbit IgG as a second biomaterial was flown onto the SPR sensor chip at a flow rate of 50 μl/min. As a result, the SPR angle after flowing the PBS solution was changed by 0.02°. This indicates that the rabbit IgG was bound on the surface of the SPR sensor chip by the immune reaction.

### Example 7

### Recovery of an Antigen and an Antibody by a Biomaterial Recovery Instrument

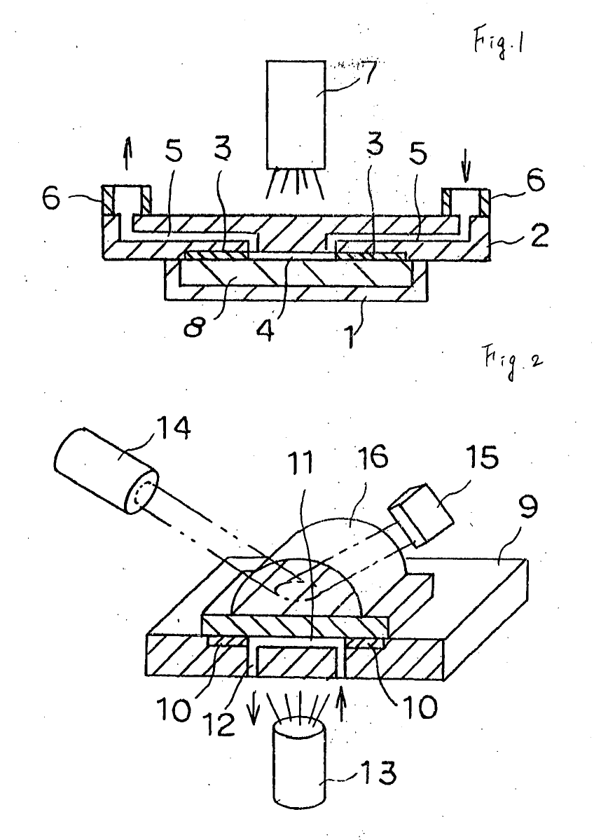

After measuring the SPR angle as described above, the SPR sensor chip was removed from the SPR measurement instrument, and was attached to a biomaterial recovery instrument described below, as shown in FIG. 1. Then, with irradiating light of 10 mW/cm² at a wavelength of 470 nm on the surface of the SPR sensor chip, PBS was flown at a flow rate of 50 μl/min on the surface and then was recovered. The recovered PBS was analyzed to confirm that it contained a sum of 1 ng of the goat-derived anti-rabbit IgG antibody and the rabbit IgG.

### Example 8

### SPR Measurement-Recovery Complex Instrument and Recovery of an Antibody Only

An SPR measurement-recovery complex instrument described below was constituted as shown in FIG. 2. Rabbit IgG was bound onto an SPR sensor chip which immobilized goat-derived anti-rabbit IgG antibody in the same manner as described in Example 6. 100 μl of 10 mM hydrochloric acid was flown onto the SPR sensor chip at a flow rate of 50 μl/min to remove the rabbit IgG from the surface of the SPR sensor chip. Subsequently, PBS was flown onto the SPR sensor chip at a flow rate of 50 μl/min for 5 minutes to adjust pH. Then, irradiation light was changed, and PBS was flown at a flow rate of 50 μl/min on the surface with irradiating light of 10 mW/cm² at a wavelength of 470 nm on the surface of the SPR sensor chip. 40 μl of the recovered PBS was analyzed to confirm that it contained 1 ng of the goat-derived anti-rabbit IgG antibody, but did not contain the rabbit IgG.

### Example 9

### Biomaterial Recovery Instrument

A biomaterial recovery instrument as shown in FIG. 1 is provided with a supporting member 1 of saucer-shape in which the upper part is open, and a recovery member 2 assembled to closely adhere to the top of the upper part of the member 1. The recovery member 2 is made of a suitable transparent material such as glass and transparent plastics, and in the central base part, a recovery space 4 is opened which is surrounded by a seal member 3 of a soft material such as rubber or soft plastics. The recovery space 4 is connected to one pair of liquid-transporting tubes 6 in the upper part through a flow path 5. Further, the recovery space 4 leads to the top of the open part of the supporting member 1 when the supporting member 1 and the recovery member 2 are assembled as described above. A photo-irradiator 7 is equipped above the recovery member 2.

An SPR sensor chip 8, which is suitable for use in the biomaterial recovery instrument shown in FIG. 1 and immobilizes biomaterials such as a complex of a goat-derived anti-rabbit IgG antibody and an antigen thereof (it may be also a photo-immobilizing carrier for a biomaterial not containing a thin metal film), is fitted into the supporting member 1 of saucer-shape with the face for photo-immobilizing the biomaterial being on the top, and the recovery member 2 is closely adhered on the face. With this setting, the biomaterial-immobilizing area is located facing the recovery space 4. Then, if a recovering solution such as a buffer solution is flown at a constant flow rate in one direction by a liquid-transporting actuator not shown in Figures via a pair of the liquid-transporting tubes 6 while carrying out photo-irradiation by the photo-irradiator 7, the biomaterial immobilized on the SPR sensor chip 8 is isolated and recovered in the recovered solution.

### Example 10

### SPR Measurement-Recovery Complex Instrument

An SPR measurement-recovery complex instrument shown in FIG. 2 has a recovery space 11 which is open and surrounded by a seal member 10 of a soft material such as rubber or soft plastics in the central surface part (upper face of the figure) of a substrate member 9 made of a suitable transparent material such as glass and transparent plastics. A recovery space 11 is connected to a liquid-transporting tube not shown in Figures via a flow path 12. A photo-irradiator 13 used for photo-immobilization and recovery of the biomaterial is equipped under the substrate member 9. A photo-irradiator 14 for measuring SPR and a photo-receiver 15 are equipped above the substrate member 9 along the reflection angle of light.

For use of the SPR measurement-recovery complex instrument shown in FIG. 2, the SPR sensor chip 8 is placed in the central surface part of the substrate member 9 (the open part of the recovery space 11) with the face for photo-immobilizing the biomaterial being on the lower side, and the biomaterial-immobilizing area is faced with the open part of the recovery space 11. Then, with flowing a solution containing a goat-derived anti-rabbit IgG antibody onto the surface of the SPR sensor chip 8 (the lower face) through the flow path 12, photo-irradiation is conducted by the photo-irradiator 13 from the lower part to immobilize goat-derived anti-rabbit IgG antibody. Then, with flowing a solution containing rabbit IgG onto the surface of the SPR sensor chip 8 (lower face) through the flow path 12, an antigen-antibody complex of the immobilized goat-derived anti-rabbit IgG antibody is formed.

Then, a prism 16 of a predetermined shape is placed on the upper face of the substrate member 9 in a closely adhered state (the prism 16 may be also placed in advance), and SPR measurement is carried out by photo-irradiation with a photo-irradiator 14 for the SPR measurement and by receiving the reflected light in the interface between the prism 16 and the substrate member 9, with the photo-receiver 15.

After completing the SPR measurement, the antigen-antibody complex immobilized on the SPR sensor chip 8 is isolated and recovered in the recovering solution by flowing the recovering solution such as a phosphate solution onto the surface of the SPR sensor chip 8 (the lower face) at a constant rate through the flow path 12, while carrying out photo-irradiation again by the photo-irradiator 13.

### Industrial Applicability

The present invention provides a method for carrying out analysis, etc. for a variety of extremely small biomaterials, wherein the method comprises immobilizing a biomaterial on a surface of a carrier, forming a complex of the biomaterial, and recovering the biomaterial or a complex thereof without damaging them, and a surface plasmon resonance sensor implementing the above method which can recover a sample with maintaining the activity or the function thereof. Therefore, the present invention is greatly useful for analysis, etc. of biomaterials.

1. A method for photo-immobilizing and/or recovering a biomaterial comprising:
(a) a process of immobilizing the biomaterial by placing the biomaterial on a surface of a carrier wherein a photo-immobilizing material plasticized by photo-irradiation and having immobilization ability by photo-modification is used at least in a surface layer part, and immobilizing the biomaterial on the carrier surface by photo-irradiation; and
(b) a process of recovering the immobilized biomaterial by isolating the immobilized biomaterial from the carrier surface by photo-irradiation and by applying external mechanical force.

2. The method according to claim 1, wherein the biomaterial is at least one selected from a group consisting of polypeptide, saccharide chain, polynucleotide, organelle, cell, bacteria, virus and a combination thereof.

3. The method according to claim 1, further comprising an intermediate process of detecting, utilizing or analyzing the immobilized biomaterial on the carrier surface between the immobilizing process and the recovering process.

4. The method according to claim 3, wherein the intermediate process comprises specifically adsorbing or reacting one kind of a second biomaterial on or with the above-mentioned biomaterial, or specifically adsorbing or reacting plural kinds of a second biomaterials sequentially on or with the above-mentioned biomaterial to form a complex, and further in the recovering process, the complex is isolated from the carrier surface and recovered.

5. The method according to claim 3, wherein the intermediate process is detecting the above-mentioned complex.

6. The method according to claim 5, wherein the above-mentioned complex is detected by a detection marker for detecting a second biomaterial which constitutes the complex.

7. The method according to claim 5, wherein a thin metal film layer is provided on the above-mentioned surface layer part of the carrier, and detection of the above-mentioned complex is carried out by detecting a resonance angle change of a surface plasmon resonance caused by a refractive index change in the surface layer part of the carrier due to the formation of the complex.

8. The method according to claim 1, wherein the above-mentioned photo-immobilizing material is a material possessing a chemical structure capable of causing cis-trans optical isomerization by photo-irradiation.

9. The method according to claim 8, wherein the chemical structure is a pigment structure having an azo group.

10. The method according to claim 1, wherein the photo-irradiation in the immobilizing process and the recovering process is photo-irradiation of photon energy which is low enough not to cause activity loss or damage of the biomaterial, and further with such power that elevation of temperature around the biomaterial is maintained within a temperature range which does not damage the function of the biomaterial.

11. The method according to claim 1, wherein the above-mentioned photo-immobilizing material has a structure comprising a component possessing a chemical structure capable of causing cis-trans optical isomerization by photo-irradiation, in a matrix material.

12. The method according to claim 11, wherein the above-mentioned component possessing the chemical structure is dispersed in the matrix material.

13. The method according to claim 11, wherein the above-mentioned component possessing the chemical structure is chemically bonded or hydrogen-bonded to the matrix material.

14. The method according to claim 11, wherein the above-mentioned matrix material is a polymer material comprising any one of a urethane group, a urea group and an amide group.

15. The method according to claim 13, wherein the above-mentioned photo-immobilizing material is any one selected from Formula I to Formula IV.
